(19) United States
(12) Patent Application Publication
(10) Pub. No.: US 2007/0239182 A1
(43) Pub. Date: Oct. 11, 2007

(54) THROMBUS REMOVAL DEVICE

(75) Inventors: Robert C. Glines, Santa Cruz, CA (US); Kevin Brounstein, Fremont, CA (US); Brendon Carver, Belmont, CA (US)

Correspondence Address: CROMPTON, SEAGER & TUFTE, LLC, 1221 NICOLLET AVENUE, SUITE 800, MINNEAPOLIS, MN, 55403-2420, US

(73) Assignee: BOSTON SCIENTIFIC SCIMED, INC., Maple Grove, MN (US)

(21) Appl. No.: 11/278,498

(22) Filed: Apr. 3, 2006

(51) Int. Cl. A61B 17/22

(52) U.S. Cl. 606/159

(57) ABSTRACT

A thrombus removal device that may include a catheter having a proximal end and a distal end, an aspiration lumen therebetween, and a guidewire lumen, a vacuum source fluidly connected to the proximal end of the aspiration lumen, a wire having a straight proximal portion and a macerating portion distal the proximal portion and disposed in the aspiration lumen, and a motor mechanically connected to the wire proximal portion to effect movement of the wire and may further include a wire having a wave-propagating portion, and methods of use thereof.

### FIELD

This invention pertains to the field of removing blockages from the lumen of body vessels and more particularly pertains to devices and methods for performing procedures such as aspiration and thrombectomy.

### BACKGROUND

Vascular procedures such as angioplasty, atherectomy, thrombectomy, stent placement, etc., used for treating occlusive vascular diseases cause material to dislodge from the inside wall of blood vessels and enter the bloodstream. The dislodged material (e.g., plaque), known as emboli, may be sufficiently large to occlude downstream blood vessels, potentially blocking blood flow to tissue. Additionally, blood clots, known as thrombi, may form and either be sufficiently large or grow over time and block a blood vessel should the thrombus become released into the bloodstream.

Numerous interventional systems and methods that employ a filtration device designed to capture debris during the treatment or diagnosis of vascular disease are well known in the art. The procedure typically involves using a filter delivery catheter to transluminally insert and deliver, within a blood vessel, a filtration device to a location distal of a target site, such as a stenosis or a lesion, and then deploying the filter causing it to open; During such filter delivery process, plaque could be liberated from the walls of the blood vessel and create emboli and thrombi that may enter the bloodstream and potentially occlude distal blood vessels.

After deploying the blood permeable filtration device distal of the target site, an interventional device may be delivered over the guide wire to the target site. During treatment of a stenosis or a lesion within the blood vessel, plaque may be liberated from the walls of the blood vessel creating emboli and thrombi. This debris could then be entrapped in the distally deployed filter, and hinder its removal from the vasculature.

In view of the foregoing, it is desirable to provide an aspirating filter delivery catheter and method for extracting debris during the filter delivery process. It is further desirable to provide an aspirating filter retrieval catheter for extracting debris accumulated proximal of the filter prior to extracting the filter from the vasculature.

It is desirable in thrombus removal catheters to maintain efficient flow rates through the aspirate lumen for the passage and removal of blood and thrombus particulate and to effectively remove thrombus particulate while maintaining an open aspirate lumen without clogging or loss of the vacuum and while maintaining the integrity of the vessel walls.

### SUMMARY

One embodiment pertains to a thrombus removal device that may include a catheter having a proximal end and a distal end, an aspiration lumen therebetween, and a guidewire lumen, a vacuum source fluidly connected to the proximal end of the aspiration lumen, a wire having a straight proximal portion and a macerating portion distal the proximal portion and disposed in the aspiration lumen, and a motor mechanically connected to the wire proximal portion to effect movement of the wire. The macerating portion may have a helical shape, a zigzag shape or other suitable shape and may define a cylindrical space to receive a thrombus. The macerating portion is located within the distal portion of the catheter. The device may include a localized power source connected to the motor and the vacuum source may be localized as well. The device may further include a wave-propagating wire such as a guidewire having a piezoelectric portion. Various configurations are contemplated, including ones having an opening between the aspiration lumen and the guidewire lumen proximal the distal end of the catheter, where the distal end of the aspiration lumen is distal the distal end of the guidewire lumen, where the distal end of the aspiration lumen is proximal the distal end of the guidewire lumen and generally includes a power source connected to the wave-propagating portion.

Another embodiment pertains to a thrombus removal device that includes a catheter having a proximal end, a distal end and a lumen defined by a wall therebetween, a vacuum source fluidly connected to the proximal end of the lumen, a wire disposed in the catheter having a wave-propagating portion, and a power source electrically connected to the wire. The wave-propagating portion may be disposed in the lumen proximal to the distal end of the catheter and may produce subsonic or supersonic waves. The wire may be at least partially embedded in the catheter wall and may form a loop on the inside of the catheter wall aspiration lumen proximate the distal end of the catheter or may be disposed on at least a portion of the aspiration lumen distal mouth. The device may also include a wire having a macerating portion disposed in the lumen.

One embodiment pertains to a method of use of one of these devices and may include the steps of providing an aspiration catheter having a lumen fluidly connected to a vacuum source, providing a wire having a macerating portion disposed in the lumen, advancing the aspiration catheter to the thrombus, providing vacuum to aspirate at least a portion of the thrombus, and operating the wire to fragment the portion of the thrombus. The aspiration catheter may include a guidewire lumen and the method may include the step of advancing the catheter over a guidewire. The macerating portion of the wire may define a cavity and the method may further include the step of drawing the portion of the thrombus into the cavity. A wire having a wave-propagating portion may be provided, and the method may include the step of activating the wave propagating portion to fragment the thrombus and the step of advancing the wave propagating portion distal of the aspiration catheter. In an alternative embodiment, the aspiration catheter may include a guidewire lumen and an opening between the guidewire lumen and the aspiration catheter proximal the distal end of the aspiration catheter and the method may include the step of advancing the wave-propagating portion to the opening. The wave-propagating portion may be a loop disposed on the lumen of the catheter proximate its distal end or may be disposed on a portion of a distal mouth of the aspiration catheter. The method may include advancing the aspiration catheter over a distal protection guide wire or use with other intravascular devices such as guide catheters and therapy catheters such as stent delivery catheters and angioplasty catheters.

### BRIEF DESCRIPTION OF THE DRAWINGS

The following detailed description may be read in reference to the drawings in which:

FIG. 1 is a diagrammatic depiction of a thrombus removal device 10;

FIGS. 2A, 2B and 2C are successive diagrammatic depictions of a thrombus removal device 34 in use in a body vessel lumen 36;

FIG. 3 is a diagrammatic cross-sectional view of a distal portion of a thrombus removal device 48;

FIG. 4 is a diagrammatic cross-sectional view of a distal portion of a thrombus removal device 56;

FIG. 5 is a diagrammatic cross-sectional view of a distal portion of a thrombus removal device 62;

FIG. 6 is a diagrammatic cross-sectional view of a distal portion of a thrombus removal device 68;

FIG. 7 is a diagrammatic cross-sectional view of a distal portion of a thrombus removal device 70;

FIG. 8A is a diagrammatic cross-sectional top view of a distal portion of a thrombus removal device 76; and

FIG. 8B is a diagrammatic cross-sectional side view of a distal portion of the thrombus removal device 76.

### DETAILED DESCRIPTION

The following description should be read with reference to the drawings, in which like elements in different drawings are numbered in like fashion. The drawings, which are not necessarily to scale, depict selected embodiments and are not intended to limit the scope of the invention. Although examples of construction, dimensions, and materials are illustrated for the various elements, those skilled in the art will recognize that many of the examples provided have suitable alternatives that may be utilized.

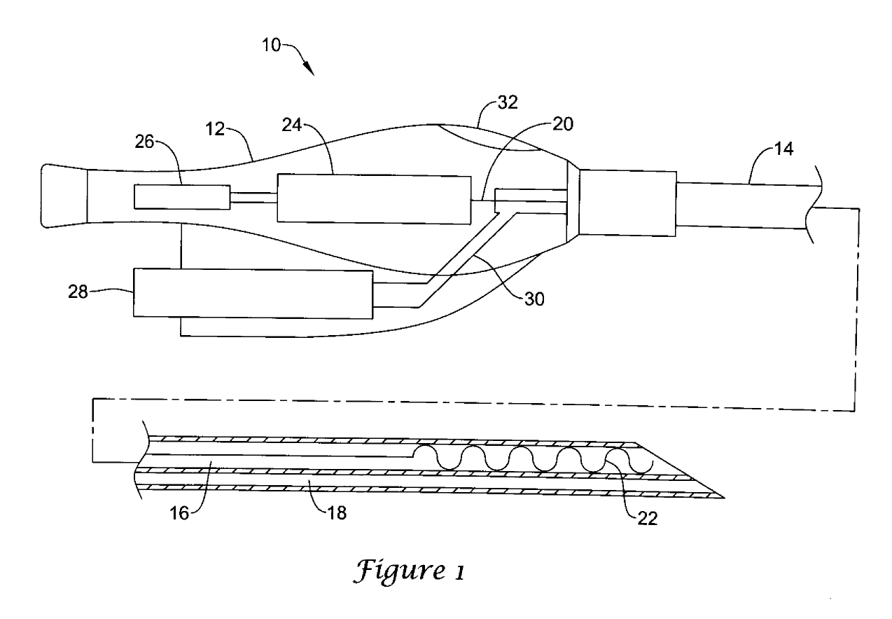

Referring now to FIG. 1, which is a depiction of a thrombus removal device 10 showing the proximal portion in a diagrammatic fashion and the distal portion in cross-section, the first embodiment will now be described. Thrombus removal device 10 has a handle portion 12 from which a catheter 14 having a main lumen 16 and a wire 20 disposed in that lumen extend in a distal direction. The wire 20 is attached proximally to an electric motor 24 powered by a power source 26 and extends proximally from lumen 16 through a seal that permits the movement of the wire while preventing passage of a fluid such as air or blood. A button 32 controls the motor. The lumen 16 is fluidly connected by a passage 30 to a vacuum source 28. Catheter 14 also includes a guide wire lumen 18. Wire 20 has a macerating portion 22 at its distal end, which substantially stays within lumen 16.

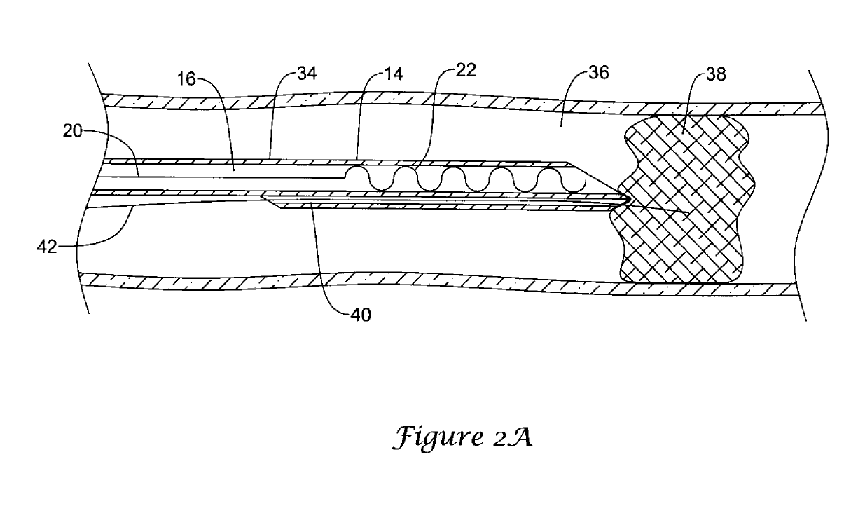

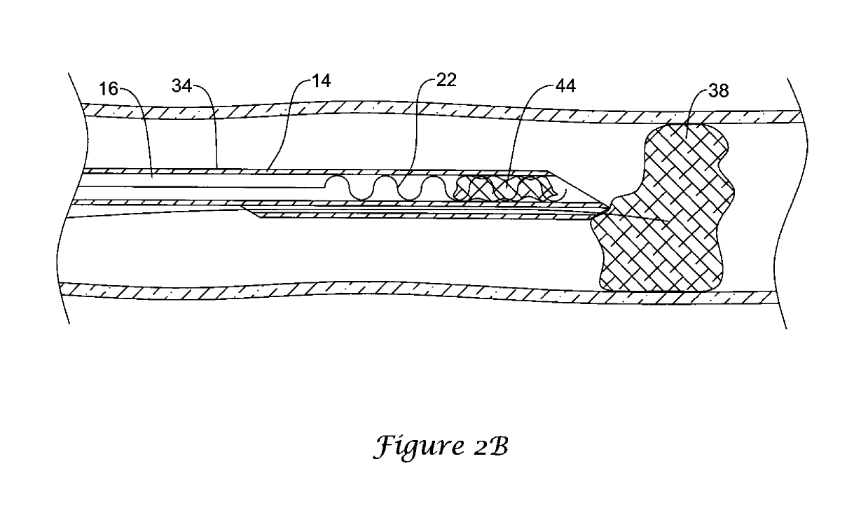

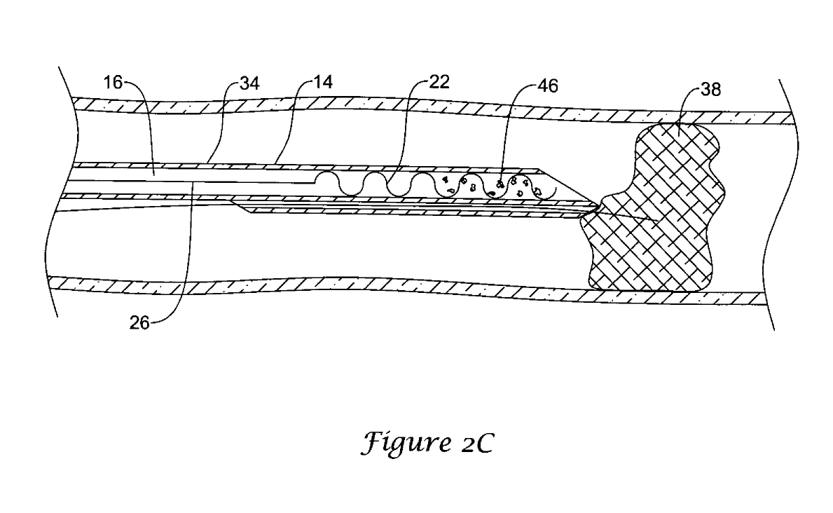

FIGS. 2A, 2B and 2C show sequential steps in an operation of a thrombus removal device 34, whose proximal portion is similar to that of device 10. A guide wire 42 is placed in the lumen 36 of a blood vessel and advanced to an area of interest containing a clot 38. Thrombus removal device 34 has a short guide wire lumen 40 by which it is advanced over the guidewire to the area of interest. Vacuum is produced in lumen 16, which as shown in FIG. 2B, may pull a fragment 44 of clot 38 into the lumen. The macerating portion 22 of the wire is rotated to reduce fragment 44 into debris 46, which may be more easily aspirated from the lumen. The rotation may be in a continuous fashion or it may be pulsed.

One embodiment, thrombus removal device 10, has been described by its parts, and another embodiment, thrombus removal device 34, has been described by its use. Thrombus removal devices whose nature and use vary from that described above are contemplated as well.

For example, handle portion 12 is shown as having a particular shape but there are many more shapes quite suitable for use as a handle and it should not be thought that thrombus removal devices described herein are limited to a particularly shaped handle. Further, all the proximal parts such as the motor and the vacuum source are shown as integrated into the handle. While such unity may be quite convenient for certain applications, it is by no means the case that such unity is required. Embodiments are contemplated where the vacuum source is a separate device and attached by a hose to the thrombus removal device, where there is no power source and the device is plugged into the wall, or where the motor is electric, hydraulic, or pneumatic or operates by other principal such as vacuum operated. Such unity is not required in another sense as well; the handle portion may be made in a fashion where it is convenient to disassemble and to replace parts. For example, the vacuum source may be a vial or syringe filled with a vacuum and whose replacement may be desired during the course of a procedure, or may be a vacuum pump or a vacuum created by air compression. The motor and the power source may be removable to provide access to the seal through which the wire passes, to allow for such adjustments as may be desired. The wire itself may be removable and replaceable or the wire may be adjustable relative to the catheter. The button may be any suitable control and it may allow for merely the turning off or on of the motor or it may be a more sophisticated control that permits one to vary the speed of the motor as desired. And of course, as the parts of the thrombus removal device vary so too may the button. It was called a button because the thrombus removal device 10 required only a simple control, but many variations of the device may have more sophisticated controls, having multiple buttons, dials and displays as may be appropriate for a particular device. Other parts may be incorporated into the device as desired. For example, a computer may be included whose function may include any or all of the following functions or any function which may enhance the device. The computer may monitor the speed of the motor, the pressure in the aspiration lumen or other useful values and it may record these values or use these values as part of a safety or diagnostic routine or it may communicate these values externally. There may be other parts, such as a torque limiter or other device interposed between the motor and the wire to limit the amount of torque the motor can transmit to the wire.

The motor 24 is an electric motor transmitting rotational motion to the wire. Motors are contemplated that, in addition to or in lieu of rotational motion, transmit oscillating longitudinal motion, subsonic or supersonic energy or other energy such as heat.

Wire 20 has macerating portion 22 that in the embodiments shown has a generally helical or spiral shape. The shape of macerating portion 22 and the type of motion imparted by the motor should be complementary, but macerating portion 22 is capable of a wide variety of suitable shapes. Macerating portion 22 may have, for example, a planar sinusoidal form or a zigzag form. It may be shaped like a whisk or have blades like a food processor. Macerating portion 22 is disposed in the distal portion of lumen 16—there may be a small gap between the distal end of macerating portion 22 and the distal opening of the lumen or there may be a portion of wire 20 that extends distally beyond macerating portion 22 or macerating portion 22 may extend proximally to the proximal end of the catheter in some applications—but the portion of wire 20 referred to as macerating portion 22 is understood to be in the distal portion of the lumen. Wire 20 may be made from stainless steel or from a nickel-titanium alloy or may be made from another biocompatible metal or alloy; it may further have a hydrophilic coating or a polymer coating. The wire may have a circular cross-section, a hollow cross-section or other suitable cross-section; it may have a uniform composition and shape along its length or it may vary as desired. For example, the cross section of macerating portion 22 may be rectangular or wedge-shaped. Macerating portion 22 may have a cross-sectional extent that is essentially the same as that of lumen 16 or it may have an extent that is substantially smaller. For example, macerating portion 22 may have a cross-sectional extent that is only 80%, 65% or 50% of that of lumen 16. In some applications, the wire may be an ultrasonic, subsonic or vibrating wire as described below.

Catheter 14 is depicted as a two-lumen catheter having one lumen 16 for aspiration and a second lumen 18 for use with a guide wire. Catheter 14 may have more or fewer lumens for particular applications. For example, catheter 14 may be a single-lumen catheter whose method of use might be as follows. The catheter is advanced to the area of interest over a guide wire, which is then withdrawn. Wire 20 is advanced through the catheter and handle portion 12 is attached to the catheter to complete the assembly of the thrombus removal device prior to operation. Catheter 14 may also have additional lumens. It may, for example, have an infusion lumen or a lumen for an angioscope or other device. Further, catheter 14 is shown as having a beveled distal end, but it may have an end of any appropriate shape.

Devices 10 and 34 are called thrombus removal devices and the blockage in the blood vessel depicted in FIG. 2 is called clot 38. It should therefore be evident that devices 10 and 34 are useful for, and contemplated for use with, more than the removal of thromboses. Throughout this specification, the terms following terms should be considered synonymous unless their particular application is clearly contrary to this general principle: thrombus, calculus, embolus, clot, blockage, organized blood product, occlusion, fragment, debris and the like. This applies to total occlusions and partial occlusions, to matter adhering to the vessel wall and to matter merely wedged between two vessel walls or free to flow in the vessel lumen. And, while the focus of this specification is on intravascular applications, the removal devices described may be used in other vessel lumens and the terms used to describe such blockages as may be found in these other vessel lumens may be appended to the above list for these particular applications. Such applications may include aspiration, thrombectomy, drug-delivery, neurovascular, electrophysiology, endoscopy, oncology, urology, and gynecology. Thus the thrombus removal devices described above may be thought of equally as occlusion removal devices or emboli removal devices.

Guide wire 42 may be a typical guide wire or may be a distal protection guide wire, having a distal protection device such as an expandable filter or balloon on the end. Any elongate member over which catheter 14 may be advanced to the area of interest may be suitable for use with the thrombus removal device.

Such variations as described above may also be used with the embodiments described below.

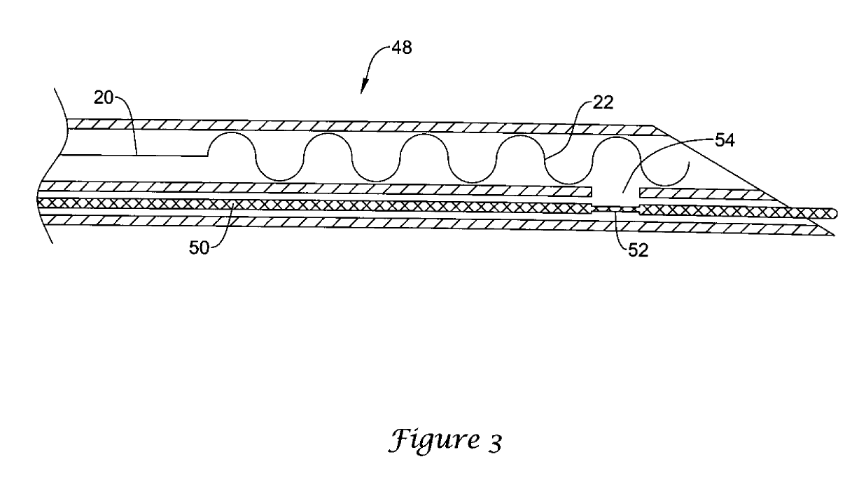

FIG. 3 is a cross-sectional diagrammatic view of the distal portion of a thrombus removal device 48 which differs from that of thrombus removal device 10 in having an opening 54 between the aspiration lumen and the guide wire lumen. This embodiment includes a guide wire 50 having a wave-propagating portion 52 that emits waves of a subsonic or ultrasonic frequency. Such waves may aid in the fragmentation of the thrombus. One such guide wire 50 may be made by connecting the portion 52 to a voltage source with an electrical conduit extending through the guide wire and coating the portion 52 with piezoelectric crystals. The guide wire 50 is fashioned such that wave-propagating portion 52 is aligned with opening 54. The guide wire may be fixed in place or may be slidable within the lumen. If it is slideable within the lumen, it may include markings or other means at the distal end to enable one to determine when the portion 52 is aligned with the opening. Alternatively, there may be radiopaque markings around the opening and in the guidewire or there may be a stop on the guidewire or other means to align it with the opening. In use, the wave-propagating portion may be activated during aspiration and while the wire 20 is rotated to aid in the fragmentation of any thrombus.

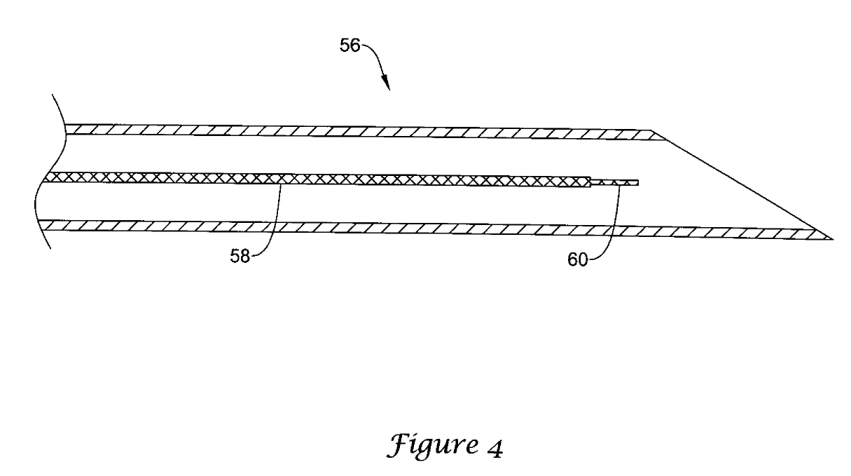

FIG. 4 is a cross-sectional diagrammatic view of the distal portion of a thrombus removal device 56, which includes an aspiration catheter with a guidewire 58 having a wave-propagating portion 60. The wave-propagating portion 60 is disposed within the distal portion of the catheter.

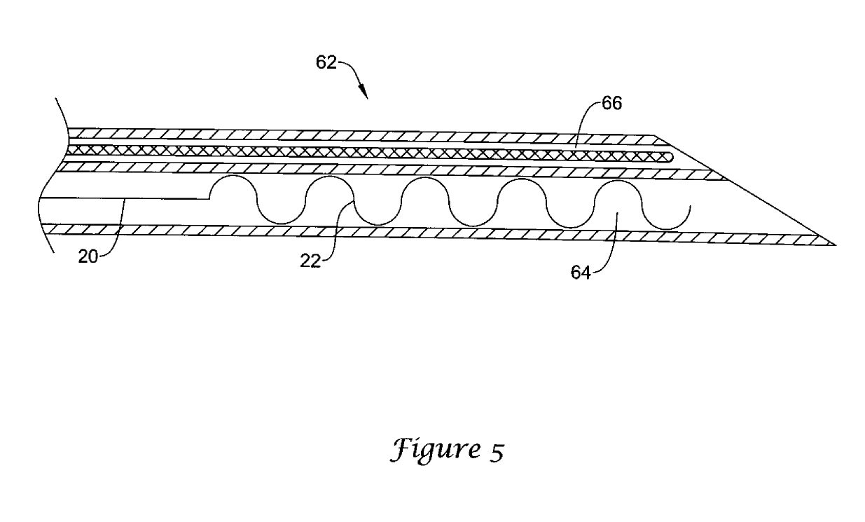

FIG. 5 is a cross-sectional diagrammatic view of the distal portion of a thrombus removal device 62 illustrating an alternate configuration of guide wire lumen 66 and aspiration lumen 64, where the distal opening of aspiration lumen 64 is distal the distal opening of guidewire lumen 66.

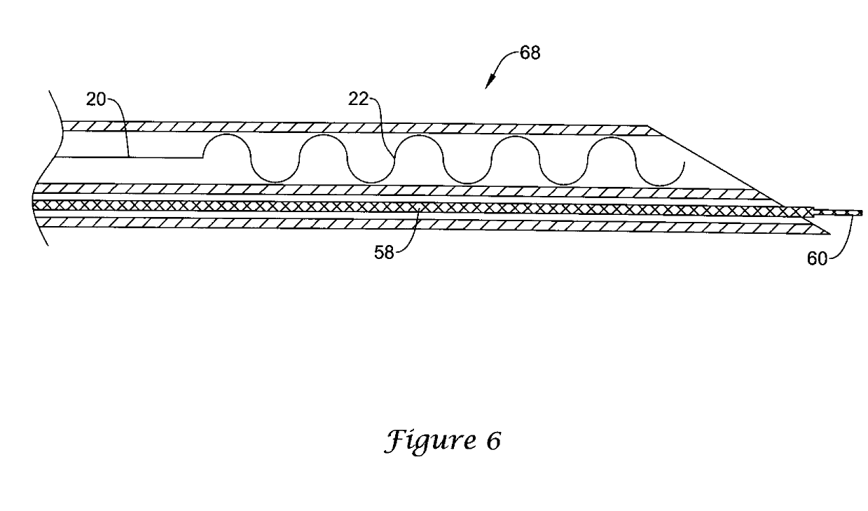

FIG. 6 is a cross-sectional diagrammatic view of the distal portion of a thrombus removal device 62 illustrating a configuration where a guide wire 58 having a wave-propagating portion 60 may be advanced distally of the catheter. The guide wire 58 may be used to weaken the thrombus in the blood vessel while wire 20 may be used to fragment portions aspirated into the aspiration lumen.

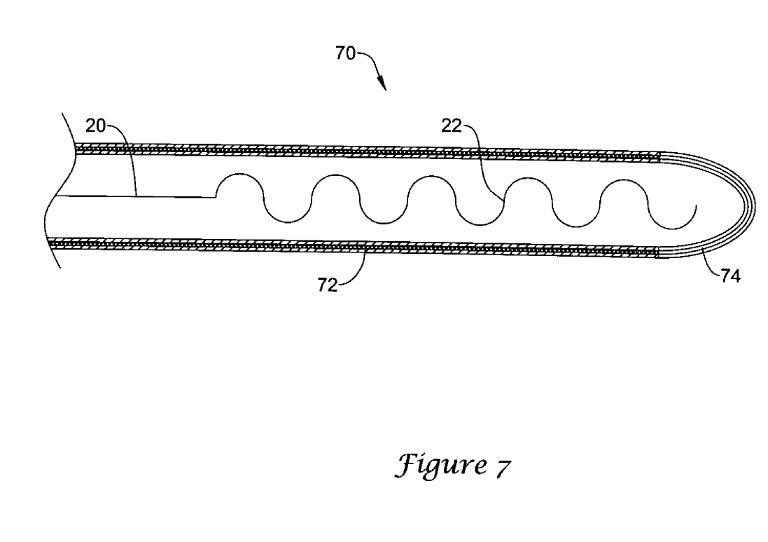

FIG. 7 is a cross-sectional top view of the distal portion of a thrombus removal device 62. Like the catheters depicted in other embodiments, the catheter of this embodiment has a beveled distal end, the distal half of which may be seen in this figure. A wire 72 is embedded in the catheter and has a distal portion 74 disposed in a groove or on the catheter wall of the distal end. This distal portion 74 is a wave-propagating portion similar to those described above.

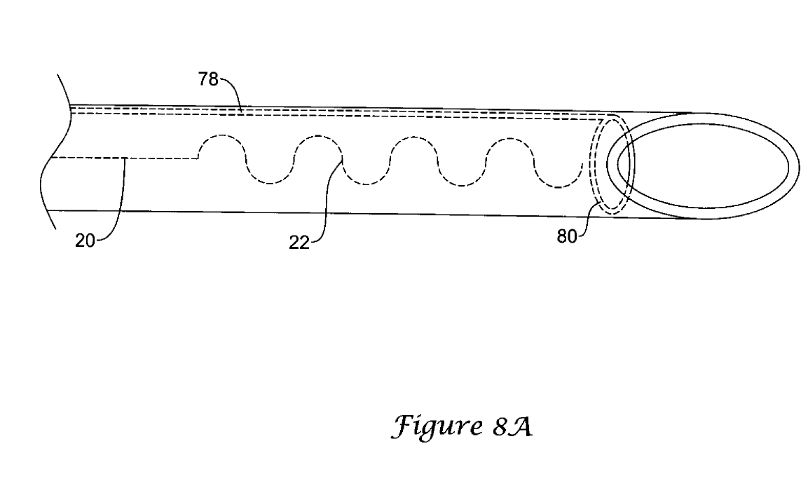

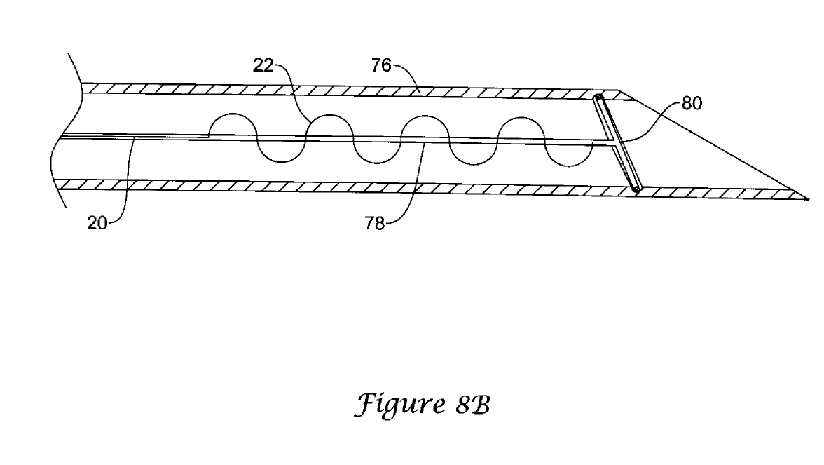

FIGS. 8A and 8B are, respectively, a top diagrammatic view and a side cross-sectional diagrammatical view of the distal portion of a thrombus removal device 76. Device 76 has a wire 78 having a wave-propagating loop 80 disposed in a groove in the catheter wall.

Numerous advantages of the invention covered by this document have been set forth in the foregoing description. It will be understood, however, that this disclosure is, in many respects, only illustrative. Changes may be made in details, particularly in matters of shape, size, and arrangement of parts without exceeding the scope of the invention. None of the description in the present application should be read as implying that any particular element, step, or function is an essential element which must be included in the claim scope. Moreover, none of these claims are intended to invoke 35 U.S.C. § 112, ¶ 6 unless the exact words “means for” are followed by a participle. The invention's scope is, of course, defined in the language in which the appended claims are expressed.

What is claimed is:

1. A thrombus removal device comprising
a catheter having a proximal end and a distal end, an aspiration lumen therebetween, and a guidewire lumen;
a vacuum source fluidly connected to the proximal end of the aspiration lumen;
a wire having a straight proximal portion and a macerating portion distal the proximal portion and disposed in the aspiration lumen; and
a motor mechanically connected to the wire proximal portion to effect movement of the wire.

2. The device of claim 1 wherein the macerating portion has a helical shape.

3. The device of claim 1 wherein the macerating portion has a zigzag shape.

4. The device of claim 1 wherein the macerating portion is proximal the distal end of the catheter.

5. The device of claim 1 wherein the macerating portion of the wire defines a cylindrical space to receive a thrombus.

6. The device of claim 1 further comprising a localized power source connected to the motor.

7. The device of claim 1 wherein the vacuum source is a localized vacuum source.

8. The device of claim 1 further comprising a wave-propagating guidewire.

9. The device of claim 1 further comprising a guidewire having a piezoelectric portion.

10. The device of claim 9 further comprising an opening between the aspiration lumen and the guidewire lumen proximal the distal end of the catheter.

11. The device of claim 1 wherein the distal end of the aspiration lumen is distal the distal end of the guidewire lumen.

12. The device of claim 1 wherein the distal end of the aspiration lumen is proximal the distal end of the guidewire lumen.

13. The device of claim 8 further comprising a power source electrically connected to the guide wire.

14. A thrombus removal device comprising:
a catheter having a proximal end, a distal end and a lumen defined by a wall therebetween;
a vacuum source fluidly connected to the proximal end of the lumen;
a wire disposed in the catheter having a wave-propagating portion; and
a power source electrically connected to the wire.

15. The thrombus removal device of claim 14 wherein the wave-propagating portion is disposed in the lumen proximal to the distal end of the catheter.

16. The device of claim 14 wherein the wave-propagating portion produces subsonic waves.

17. The device of claim 14 wherein the wave-propagating portion produces supersonic waves.

18. The device of claim 14 wherein the wire is at least partially embedded in the catheter wall.

19. The device of claim 18 wherein the wave-propagating portion forms a loop on the inside of the catheter wall.

20. The device of claim 18 wherein the lumen has a mouth at the catheter distal end and wherein the wave-propagating portion is disposed on at least a portion of the mouth.

21. The device of claim 20 wherein the wave-propagating portion is disposed on a distal portion of the mouth.

22. The device of claim 14 further comprising a wire having a macerating portion, wherein the macerating portion is disposed in the catheter lumen.

23. A method of removing a thrombus from a blood vessel, comprising the steps of:
providing an aspiration catheter having a lumen fluidly connected to a vacuum source;
providing a wire having a macerating portion disposed in the lumen;
advancing the aspiration catheter to the thrombus;
providing vacuum to aspirate at least a portion of the thrombus; and
operating the wire to fragment the portion of the thrombus.

24. The method of claim 23 wherein the aspiration catheter includes a guidewire lumen and further comprising the step of advancing the catheter over a guidewire.

25. The method of claim 23 wherein the macerating portion of the wire defines a cavity and further comprising the step of drawing the portion of the thrombus into the cavity.

26. The method of claim 23 further comprising the steps of:
providing a wire having a wave-propagating portion; and activating the wave propogating portion to fragment the thrombus.

27. The method of claim 26 further comprising advancing the wave propagating portion distal of the aspiration catheter.

28. The method of claim 26 wherein the aspiration catheter comprises a guidewire lumen and an opening between the guidewire lumen and the aspiration catheter proximal the distal end of the aspiration catheter and wherein the wave-propagating portion is advanced to the opening.

29. The method of claim 26 wherein the wave-propagating portion is a loop disposed on the lumen of the catheter proximate its distal end.

30. The method of claim 26 wherein the wave-propagating portion is disposed on a portion of a distal mouth of the aspiration catheter.

31. The method of claim 23 further comprising advancing the aspiration catheter over a distal protection guide wire.
